(19) United States
(12) Patent Application Publication
(10) Pub. No.: US 2024/0115186 A1
(43) Pub. Date: Apr. 11, 2024

(54) METHOD FOR CLASSIFYING ATRIAL FIBRILLATION

(71) Applicant: CathVision ApS, Kopenhagen (DK)

(72) Inventors: Rune PAAMAND, Humlebaek (DK); Mads Emil MATTHIESEN, Genarp (SE)

(21) Appl. No.: 18/265,504

(22) PCT Filed: Aug. 20, 2021
(86) PCT No.: PCT/EP2021/073107

§ 371 Date: Jun. 6, 2023

(30) Foreign Application Priority Data
Dec. 7, 2020 (EP) 20212255.2

(51) Int. Cl. A61B 5/361; A61B 5/00; A61B 5/308

(52) U.S. Cl. CPC A61B 5/361; A61B 5/308; A61B 5/4842

(57) ABSTRACT

The invention relates to a method for classifying atrial fibrillation by analysing ECG data (1) via a control system (2), wherein in a decomposition step the control system (2) decomposes the ECG data (1) by a frequency decomposition thereby generating frequency data (7), in particular a frequency spectrum, comprising a frequency dimension (f) and an amplitude dimension (a). It is proposed that in an analysis step the control system (2) identifies in the frequency data (7) a dominant window (8) having a width (9) in the frequency dimension (f) and meeting an amplitude criterion, that the amplitude criterion is based on a sum of amplitudes inside the dominant window (8), that the control system (2) identifies the dominant window (8) by searching on at least a section, in particular a pre-defined section, of the frequency dimension (f) for a window fulfilling the amplitude criterion and that based on the dominant window (8) the control system (2) determines the classification of atrial fibrillation in the ECG data (1).

### CROSS-REFERENCE TO RELATED APPLICATIONS

This application is a U.S. national stage of International Application Number PCT/EP2021/073107, filed Aug. 20, 201, and claims priority to EP 20212255.2, filed Dec. 7, 2020.

### BACKGROUND OF INVENTION

### Field of Invention

The invention relates to a method for classifying atrial fibrillation by analyzing ECG data via a control system and a control system for performing such a method.

### Brief Description of Related Art

Electrically, atrial fibrillation is the chaotic activation of muscle cells of the atria. During atrial fibrillation, the atria only minimally contribute to the function of the heart. Atrial fibrillation, therefore, reduces the output of the heart but is not imminently dangerous. However, when becoming chronical, atrial fibrillation is correlated with increased morbidity and mortality. One treatment option for atrial fibrillation is ablation therapy. Ablation therapy is the destruction of the cells that allow electrical wave reentry to reduce chaotic activation of the atrial muscle cells.

The success rate of and strategy for atrial fibrillation ablation heavily depends on the level of complexity of the electrical conduction through the atria. If complexity is low, ablation of the usual reentry paths has a high chance of success. If complexity is higher, additional electrical mapping of activation paths can be used. Independent of the treatment, classifying the complexity of atrial fibrillation allows patient-specific treatment and outcome prediction of ablation therapy.

One of the promising methods for classifying atrial fibrillation is calculating the dominant frequency of the electrical activation of the atria from a body surface ECG. Generally said, dominant frequency determination often uses the steps of isolating atrial activation from ECG data and analyzing a frequency spectrum of the remaining atrial signal data. Known methods (U.S. Pat. No. 5,772,604 A) detect a peak frequency in the spectrum of the atrial signal data. While these methods yield promising results, there is still room for improving the classification of atrial fibrillation by providing new classification methods.

It is therefore an object of the present invention to provide an improved method for classifying atrial fibrillation yielding more robust results.

### BRIEF SUMMARY OF THE INVENTION

The above-noted problem is solved by a method as disclosed herein.

The main realization of the present invention is that atrial activation is not adequately described by a single peak or main frequency but in itself a complex, instable and rapidly changing chaotic phenomenon. Looking for a single dominant frequency is therefore not an optimal way of describing the physiological reality. The central realization in the present case is that atrial fibrillation can be characterized by searching for a window of frequencies representing the dominant part of the atrial fibrillation.

After decomposing the ECG data thereby generating frequency data comprising a frequency dimension and an amplitude dimension, a dominant window can be found by searching in the frequency dimension for a window meeting an amplitude criterion based on a sum of amplitudes inside the dominant window.

In this way, it becomes possible for a control system to directly search for a part of the frequency dimension, the dominant window, which in particular contains a pre-defined part of the signal power, instead of looking for a single frequency with the maximum power. This method is more robust against random and/or temporary peaks of power in less relevant frequencies.

In detail, it is proposed that in an analysis step the control system identifies in the frequency data a dominant window having a width in the frequency dimension and meeting an amplitude criterion, that the amplitude criterion is based on a sum of amplitudes inside the dominant window, that the control system identifies the dominant window by searching on at least a section, in particular a pre-defined section, of the frequency dimension for a window fulfilling the amplitude criterion and that based on the dominant window the control system determines the classification of atrial fibrillation in the ECG data.

While it is known from previous methods to look for a window around the found dominant frequency, presently the window is found by searching on at least a section of the frequency dimension and not by defining the window around a dominant frequency.

In a preferred embodiment, ventricular components are removed from the ECG data to focus the analysis on fibrillation waves. In another embodiment, the amplitude dimension is an energy density or power density dimension. The frequency distribution of the power of the atrial fibrillation signal inside the ECG is directly related to the complexity of the atrial fibrillation. The same is true for the mathematically dependent signal energy.

Preferred frequency decompositions include a Fourier transformation, Welch method, a model-based decomposition, which is preferably an autoregressive moving average based decomposition. All of these methods for frequency decomposition have different strengths and can be used in varying embodiments of the proposed method. The Fourier transformation and the Welch method impose less restrictions on the signal to be analyzed compared to other methods and are therefore also well suited for analyzing the chaotic atrial fibrillation waves. Model-based decompositions like the autoregressive moving average based decomposition again provide the possibility to adapt the decomposition to expected atrial fibrillation waves.

As atrial fibrillation has components that vary rapidly over time, in another preferred embodiment, using a time-frequency decomposition is proposed. In this way, it becomes possible to separate time-stable and non-stable components of the atrial fibrillation waves. In particular, the wavelet transformation allows specifically choosing a wavelet according to expected fibrillation waves and adapting the coarseness of the transformation specifically to the purpose of searching for a dominant window in a section of the frequency dimension.

In one embodiment, the amplitude criterion may comprise a primary criterion that a sum of amplitudes in the dominant window is at least equal to a percentage of a sum of amplitudes along a part of the frequency dimension. Preferably in this way, the dominant window can be defined as a window containing a pre-defined percentage of the total power of the signal. However, other mathematically related concepts are equally preferred. For example, the power density spectrum could be a normalized or otherwise adapted power density spectrum. In this way, instead of searching for a single power spike, a search for a power band is conducted.

The width of the dominant window can be defined by using a boundary condition. In this way, further information about the signal can be included in the dominant window. For example, a small dominant window containing a large amount of power may be indicative for a less complex atrial fibrillation.

It may be useful or necessary to identify a single dominant frequency for classifying atrial fibrillation. According to an embodiment, a dominant frequency can be identified based on the dominant window.

Depending on the algorithm used for defining the width of the dominant window, it may be advantageous to analyze the width of the dominant window and classify the atrial fibrillation based on this analysis. Mostly a wider dominant window implies higher complexity of the atrial fibrillation. Further analysis of the dominant window may include looking at harmonics of the frequency values of the dominant window as harmonic dominant windows. In particular, the power contained in these harmonic dominant windows may be related to atrial fibrillation complexity.

Further preferred ways of analyzing the frequency data inside the dominant window may include analyzing skewness and/or kurtosis. Skewness and kurtosis contain additional information about the primary frequencies of atrial fibrillation and their amplitude.

According to an embodiment, the classification of atrial fibrillation may comprise a presence or absence and preferably a severity indicator of the atrial fibrillation. In particular, the seventy of atrial fibrillation is an important factor for deciding for or against ablation therapy and the used ablation method.

The electrical measurement environment includes the preferred possibility of using a body-surface ECG. Body-surface ECG data combines high precision with low invasion.

Particularly useful pre-processing steps for the proposed method include removing noise with non-linear filtering and removing powerline interference data. The, preferably subsequent, ventricular component removal step may comprise a QRS or QRST removal. Either may be combined with a morphology grouping to adapt to varying QRST complex morphology. These variations in QRST complex morphology can for example be caused by the atrial fibrillation itself. As atrial fibrillation inhibits the physiological electrical conduction from the sinus node to the ventricles, ventricular activity during atrial fibrillation may have different physiological causes resulting in two or more sharply different morphology groups each related to their physiological cause.

Further information on the atrial fibrillation may be inferred from additional analysis of the time domain.

Another teaching, which is of equal importance, is directed to a control system for performing the proposed method. All explanations given with respect to the proposed method are fully applicable.

### BRIEF DESCRIPTION OF THE DRAWINGS

In the following, an embodiment of the invention is explained with respect to the drawings. The drawings show in:

FIG. 1, steps of the proposed method applied in the time domain; and

FIG. 2, a frequency spectrum of the ECG data.

### DETAILED DESCRIPTION OF THE INVENTION

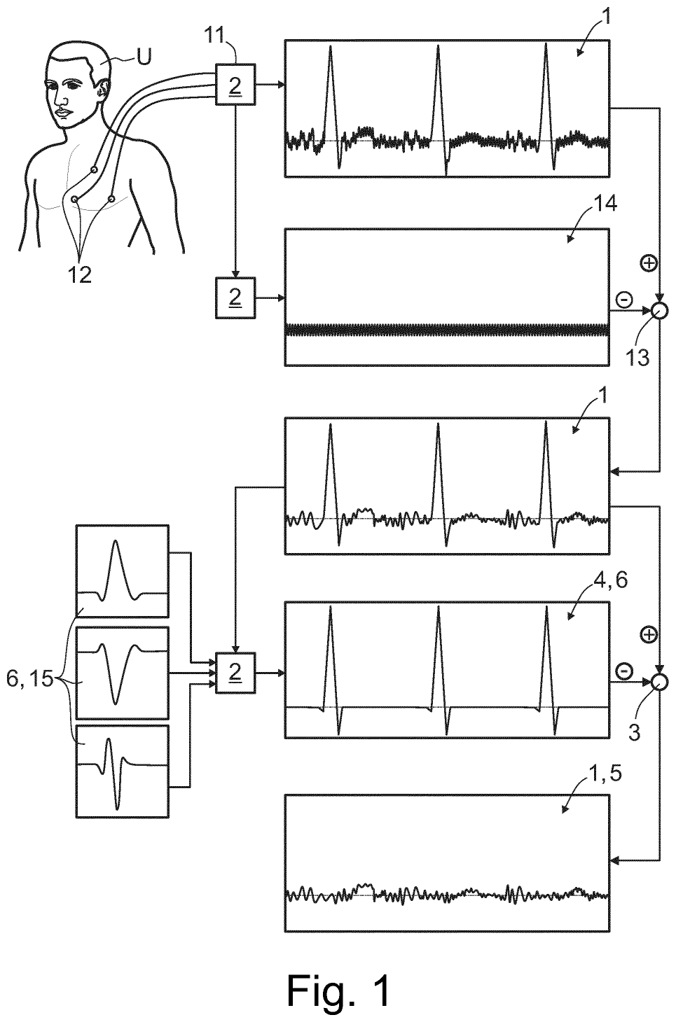

The proposed method is used for classifying atrial fibrillation by analyzing ECG data 1 via a control system 2. In FIG. 1 the control system 2 is shown schematically as a control system 2 comprising hardware connected to a user U. In this case, the control system 2 measures the ECG data 1 as a single-channel ECG. However, the ECG data 1 may also comprise one or more channels of a multi-channel ECG. Physically measuring the ECG data 1 is not necessarily comprised by the proposed method, such that the control system 2 may also process ECG data measured by another system separate from the control system 2. The control system 2 therefore also may be a general-purpose computer, cloud-based or the like, and configured to perform the proposed method.

In a preferred embodiment as shown in the figures, the method may comprise a ventricular component removal step 3 in which the control system 2 processes the ECG data 1 to remove ventricular components 4 from the ECG data 1. The ventricular components 4 may be mostly QRS or QRST complex data. As the more powerful part of the activation of the heart, removing the ventricular components 4 from the ECG data 1 allows focusing the analysis onto atrial signal data 5 contained in the ECG data 1. FIG. 1 shows the ventricular component removal step 3 in the bottom part. The top part of FIG. 1 refers to a pre-processing of the ECG data 1 which is explained further below. In this case, the ventricular component removal step 3 comprises a subtraction of a template 6. This will also be explained further below. It is equally preferred to use different methods of removing some or all ventricular components 4. In particular, the ventricular component removal step may comprise the complete removal of sections of the ECG data 1 containing the QRS or QRST complex. The method may then comprise analyzing one or more sections of the ECG data between QRS or QRST complexes. To acquire higher frequency resolution, known methods like zero padding or known methods for connecting the signal sections may be used.

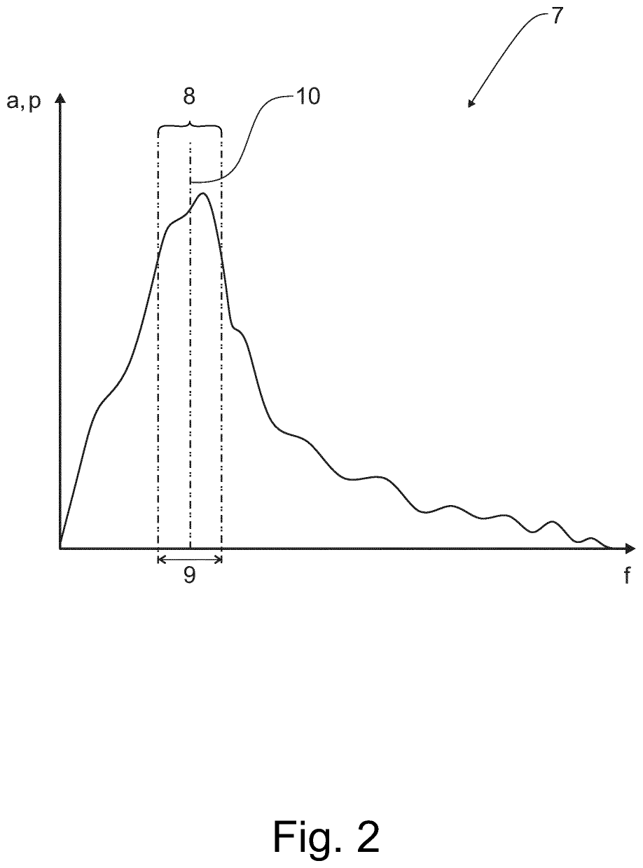

As a further step, preferably subsequent step to the removal step 3, the method comprises a decomposition step which, due to its abstract nature, is not shown in FIG. 1. In the decomposition step, the control system 2 decomposes the ECG data 1 by a frequency decomposition thereby generating frequency data 7 shown in FIG. 2. The frequency data 7 may in particular be a frequency spectrum as shown. The frequency data 7 comprises a frequency dimension f and an amplitude dimension a. The frequency decomposition may be any suitable frequency decomposition. The frequency dimension f may be separated into scales instead of “frequencies” according to the narrow physical meaning. The amplitude dimension a may represent a physical parameter like power density but may also be a purely theoretical dimension.

It is essential that in an analysis step, which due to its abstract nature is also not shown in FIG. 1 as such, the control system 2 identifies in the frequency data 7 a dominant window 8 having a width 9 in the frequency dimension f and meeting an amplitude criterion. The dominant window 8 is a part of the frequency dimension f found by looking for the amplitude criterion. The width 9 of the dominant window 8 may be pre-defined or variable. The dominant window 8 includes at least two, preferably at least 5, more preferably at least 10, frequencies or frequency bins in the frequency dimension f.

It is further essential, that the amplitude criterion is based on a sum of amplitudes inside the dominant window 8. Here and in the following the term “sum” includes calculating an integral along the frequency dimension f even though the amplitudes will usually be defined discreetly. It may also comprise any other mathematical concept leading to a value representing the sum, like calculating a mean value and the like. In the preferred embodiment, the sum of amplitudes is based on a real addition of the amplitudes and/or is the sum of all amplitudes inside the dominant window 8.

It is further essential, that the control system 2 identifies the dominant window 8 by searching on at least a section, in particular a pre-defined section, of the frequency dimension f for a window fulfilling the amplitude criterion. The search may be conducted on the whole frequency dimension f. The control system 2 may use any suitable search method. The search method may be optimized for the amplitude criterion to avoid more complex calculations on parts of the frequency dimension f that are easily excludable. The pre-defined section of the frequency dimension f may, for example, exclude non-physiological frequencies, for example, frequencies higher than 10 Hz.

It is further essential that based on the dominant window 8 the control system 2 determines the classification of atrial fibrillation in the ECG data 1.

Here and preferably, the amplitude dimension a is an energy density or power density dimension p. This dimension may be normalized or otherwise processed without changing its information content. In the embodiment shown in FIG. 2 and preferred as such, the frequency data 7 comprises a power spectral density spectrum of the ECG data 1.

The frequency decomposition may additionally or alternatively comprise a Fourier transformation and/or a Welch method. The Fourier transformation and the Welch method are well known to the person skilled in the art. The frequency decomposition may also comprise a model-based decomposition, preferably an autoregressive moving average based decomposition. Such a model-based decomposition can be adapted specifically to the atrial signal data 5. It is evident that presently a broad definition of “frequency” is used, including scales, in particular Wavelet scales, and the like.

The frequency decomposition may comprise a time-frequency decomposition with a time dimension, in particular a short-time Fourier transformation. The time-frequency decomposition may also comprise a wavelet transformation, preferably a wavelet transformation based on a Gaussian wavelet, more preferably a first or second derivative of a Gaussian wavelet. The wavelet transformation may be scaled according to an expected dominant window 8. It may also be applied in different scales subsequently during searching for the dominant window 8.

By calculating multiple frequency decompositions along the time dimension of the ECG data 1 rapidly changing transient components of the atrial activation can be identified and analyzed and/or removed. For the frequency-decomposition, in particular the time-frequency decomposition, a window function, in particular, a Hamming window, may be used. The time-frequency decomposition may be, at least partially, averaged over the time dimension.

With regards to FIG. 2, the amplitude criterion in the preferred embodiment will now be explained. The amplitude criterion here and preferably comprises the primary criterion that a sum of amplitudes in the dominant window 8 is at least equal to a percentage of a sum of amplitudes along a part of the frequency dimension f. This can be better understood by way of example. Here, the dominant window 8 is the smallest window comprising for example 15 percent of the total power of the power spectral density spectrum of the ECG data 1.

Based on this example but speaking generally, the percentage of the sum of amplitudes may be pre-defined. It may be constant, for example always 15 percent but may also depend on the ECG data 1, in particular, the atrial fibrillation data 5 and then, for example, the total power of the power spectral density spectrum. It may be mentioned again that in a continuous spectrum like the one shown in FIG. 2 the sum could be an integral. The percentage of the sum of amplitudes can be a percentage of the total frequency dimension f and it may be pre-defined. As an alternative, the part of the frequency dimension f could comprise only a physiological part or any other part of the frequency dimension f chosen in particular to exclude possible problem frequencies like powerline interference frequencies or harmonics.

The primary criterion may also be that a sum of amplitudes in the dominant window 8 and harmonic frequencies of the dominant window is at least equal to a percentage of a sum of amplitudes along a part of the frequency dimension f.

The amplitude criterion may comprise a boundary condition. The boundary condition can be an absolute condition like the amplitude dropping below a predefined value or a value defined relative to the total power or a power peak or the like. Preferably the boundary condition is a differential boundary condition and preferably the boundary of the dominant window 8 is set to a local minimum.

The amplitude criterion may further or alternatively comprise the secondary criterion that the dominant window 8 is the smallest window for fulfilling the primary criterion. With all criteria, the required search precision may be reduced.

After finding the dominant window 8 the control system 2 may conduct further analysis. Preferably the control system 2 identifies a dominant frequency 10 based on and in particular in the dominant window 8 and classifies the atrial fibrillation based on the dominant frequency 10. For example, known methods to use the dominant frequency 10 may be used with this newly identified dominant frequency 10. In the embodiment shown in FIG. 2, the dominant frequency 10 is the center, defined as the middle between the boundaries, of the dominant window 8. It may alternatively be the boundary of a, in particular pre-defined, percentile of the amplitudes of the dominant window 8 or a frequency associated with an average value, in particular the mean value or the median value, of the amplitudes of the dominant window 8. As can be seen in FIG. 2, in this example the dominant frequency 10 is not the peak frequency, leading to a different result than known algorithms.

In general, the control system 2 may carry out a plausibility check for the dominant window 8 and/or the dominant frequency 10. This plausibility check comprises checking if the dominant window 8 and/or the dominant frequency 10 are inside a physiologically plausible frequency band. If the plausibility check is negative, the control system 2 may resort to a different search method and in particular identify a second-best candidate as the dominant window 8.

In the cases where the width 9 of the dominant window 8 is not pre-defined as a constant, it may be the case that the control system 2 analyzes the width 9 of the dominant window 8 and classifies the atrial fibrillation based on this analysis. Reverting to the example, if the dominant window 8 is the smallest window fulfilling the amplitude criterion, a small dominant window 8 may be indicative for low complexity of atrial fibrillation.

The control system 2 can identify harmonic dominant windows as multiples of the frequency values of the dominant window 8 and here and preferably analyzes the harmonic dominant windows and classifies the atrial fibrillation based on the harmonic dominant windows. For example, a high power density inside the harmonic dominant windows could be used as an indicator for a good dominant window 8 estimation. Not being able to identify harmonic dominant windows could be an indicator for a high signal complexity which could be based on bad signal processing, but also on high atrial fibrillation complexity. These reasons can be separated by further analysis.

Here and preferably the control system 2 analyses skewness and/or kurtosis of the amplitude dimension a inside the dominant window 8, preferably around the dominant frequency 10, and classifies the atrial fibrillation based on the skewness and/or kurtosis.

If the method is applied to ECG data 1 of users U that have not yet been diagnosed in relation to atrial fibrillation, it may be interesting to diagnose atrial fibrillation in a first step. Hence, in such a case, the classification of atrial fibrillation may comprise a presence or absence of the atrial fibrillation. The classification of the atrial fibrillation may further or alternatively comprise a seventy indicator of the atrial fibrillation. This severity indicator may comprise a likelihood of success of ablation therapy. The method could also be used for a long time ECG to analyze the frequency of atrial fibrillation episodes and their duration. The classification may be or include a quantification of atrial fibrillation, in particular the severity indicator.

As indicated in FIG. 1, the ECG data 1 may be body surface ECG data. Other preferred embodiments include the ECG data 1 being intracardiac or subcutaneous ECG data 1. If the ECG data 1 is intracardiac data, its measurement may preferably be unipolar or bipolar.

The control system 2 may comprise an electrical input interface 11 connectable to electrodes 12 and adapted to measure the ECG data 1. In FIG. 1 there are shown only three electrodes 12 which are used to measure a single-channel ECG. However, other ECGs and in particular a 12-channel ECG may be used. The ECG data 1 can then be one of those channels or a mixture, particularly a mean value, of multiple channels. Another preferred possibility is that the electrodes 12 are part of a wearable, in particular a smartwatch, and/or a smartphone and/or an implant and/or for the ECG data 1 to be ECG data 1 measured by a wearable and/or smartphone and/or implant.

Here and preferably the control system 2 comprises electrodes 12 connected to the electrical input interface 11 and a patient, here the user U. It is preferred that the surface ECG data 1 is measured via the electrodes 12.

Also shown in FIG. 1 is a pre-processing step 13. It is preferred that in the preprocessing step 13 the control system 2 processes the ECG data 1 to remove noise, preferably by using non-linear filtering, in particular median filtering, and/or to remove powerline interference data 14, in particular at 50 Hz or 60 Hz and harmonic frequencies of 50 Hz or 60 Hz, preferably using an adaptive notch filter. As shown, the pre-processing is preferably done prior to the ventricular component removal step 3 and/or the decomposition step.

The ventricular component removal step 3 here and preferably comprises a QRS removal and preferably a T-wave filtering. The QRS removal is preferably based on the subtraction of a template 6 of a QRS complex. This template 6 may be subtracted from the ECG data 1 and the time domain. It has been found that in some instances a QRS removal is advantageous over a QRST removal. The T-wave is not always fully dependent on the QRS complex. Especially when using a template 6 for subtracting it can be advantageous to only subtract the QRS complex and filter the T-wave by a different method or with a different template 6. Here and preferably the T-wave filtering is a non-linear t-wave filtering.

In other instances, the ventricular component removal step 3 may comprise a QRST removal, preferably also based on a template 6. As morphologies of ECG data 1 vary between different persons, it may also be advantageous to implement both methods and compare the results for a single user U.

Shown in the left bottom part of FIG. 1 is the preferred embodiment that the QRS removal or QRST removal comprises a grouping of QRS or QRST complexes contained in the ECG data 1 into at least two different morphology groups, creating a QRS or QRST template 15 for at least two of the morphology groups and removing the QRS or QRST complexes of the respective morphology groups with the respective templates 15. In FIG. 1, there are shown three different templates 15. The necessary number of templates 15 may be detected automatically.

Further to the analysis described above, the control system 2 may analyze the ECG data 1 in the time domain using a time domain analysis after removing the ventricular components 4 and additionally classifies the atrial fibrillation based on the time domain analysis. Preferably the time domain analysis comprises a sample entropy analysis and/or a principle component analysis and/or a wave amplitude analysis and/or a wave correlation analysis. These may be used to generally judge the complexity of the atrial fibrillation in the time domain.

According to another teaching, a control system 2 for performing the proposed method is proposed. Reference is made to all explanations given before. The control system 2 may comprise the electrical input interface 11 and preferably electrodes 12.

### REFERENCE NUMERALS

1 ECG data
2 control system
3 ventricular component removal step
4 ventricular components
5 atrial signal data
6 template
7 frequency data
8 dominant window
9 width
10 dominant frequency
11 electrical input interface
12 electrodes
13 pre-processing step
14 powerline interference data
15 QRS template
U user
f frequency dimension
a amplitude dimension
p power density dimension

1-16. (canceled)

17. A method for classifying atrial fibrillation by analyzing ECG data via a control system,
wherein, in a decomposition step, the control system decomposes the ECG data by a frequency decomposition thereby generating frequency data having a frequency dimension and an amplitude dimension,
wherein, in an analysis step, the control system identifies in the frequency data a dominant window having a width in the frequency dimension and meeting an amplitude criterion,
wherein the amplitude criterion is based on a sum of amplitudes inside the dominant window,
wherein the control system identifies the dominant window by searching on at least a section of the frequency dimension for a window fulfilling the amplitude criterion, and
wherein, on the basis of the identified dominant window, the control system determines a classification of atrial fibrillation in the ECG data.

18. The method according to claim 17, wherein, in a ventricular component removal step, the control system processes the ECG data to remove ventricular components from the ECG data.

19. The method according to claim 17, wherein the amplitude dimension is an energy density or power density dimension.

20. The method according to claim 19, wherein the frequency data comprises a power spectral density spectrum of the ECG data.

21. The method according to claim 17, wherein the frequency decomposition comprises one or more selected from the group consisting of a Fourier transformation, a Welch method, and a model-based decomposition.

22. The method according to claim 17, wherein the frequency decomposition comprises an autoregressive moving average based decomposition.

23. The method according to claim 17, wherein the frequency decomposition comprises a time-frequency decomposition with a time dimension.

24. The method according to claim 17, wherein the time-frequency decomposition is, at least partly, averaged over the time dimension.

25. The method according to claim 17, wherein the amplitude criterion comprises a primary criterion that the sum of amplitudes in the dominant window is at least equal to and a percentage of a sum of amplitudes along part or all of the frequency dimension.

26. The method according to claim 25, wherein the amplitude criterion comprises a boundary condition and/or a secondary criterion that the dominant window is the smallest window fulfilling the primary criterion.

27. The method according to claim 17, wherein the control system identifies a dominant frequency in the dominant window and classifies the atrial fibrillation based on the dominant frequency.

28. The method according to claim 27, wherein the dominant frequency is the center of the dominant window or the boundary of a percentile of the amplitudes of the dominant window or a frequency associated with an average value of the amplitudes of the dominant window.

29. The method according to claim 17, wherein the control system analyses the width of the dominant window and classifies the atrial fibrillation based on this analysis, and/or wherein the control system identifies harmonic dominant windows as multiples of the frequency values of the dominant window and analyses the harmonic dominant windows and classifies the atrial fibrillation based on the harmonic dominant windows.

30. The method according to claim 17, wherein the control system analyses skewness and/or kurtosis of the amplitude dimension inside the dominant window and classifies the atrial fibrillation based on the skewness and/or kurtosis.

31. The method according to claim 17, wherein the classification of atrial fibrillation comprises a presence or absence and/or a seventy indicator of the atrial fibrillation.

32. The method according to claim 17, wherein the classification of atrial fibrillation comprises a seventy indicator of the atrial fibrillation, said severity indicator comprising a likelihood of success of ablation therapy.

33. The method according to claim 17, wherein the ECG data is one or more selected from the group consisting of body surface ECG data, intracardiac ECG data and subcutaneous ECG data, and/or wherein the control system comprises an electrical input interface connectable to electrodes and adapted to measure the ECG data.

34. The method according to claim 17, wherein the ECG data is body surface ECG data, wherein the control system comprises an electrical input interface connectable to electrodes, wherein the electrodes are connected to the electrical input interface and a patient, and wherein the body surface ECG data is measured via the electrodes.

35. The method according to claim 17, wherein, in a pre-processing step, the control system processes the ECG data to remove noise using non-linear filtering.

36. The method according to claim 18, wherein the ventricular component removal step comprises a QRS removal, or a QRST removal.

37. The method according to claim 36, wherein the QRS removal or QRST removal comprises grouping QRS or QRST complexes contained in the ECG data into at least two different morphology groups, creating a QRS or a QRST template for the at least two different morphology groups, and removing the QRS or QRST complexes of the respective morphology groups with the respective templates.

38. The method according to claim 18, wherein the control system analyses the ECG data in a time domain using a time domain analysis after removing the ventricular components and additionally classifies the atrial fibrillation based on the time domain analysis.

39. The method according to claim 38, wherein the time domain analysis comprises a sample entropy analysis and/or a principal component analysis and/or a wave amplitude analysis and/or a wave correlation analysis.

40. A control system for performing the method according to claim 17.
